(19) United States
(12) Patent Application Publication
(10) Pub. No.: US 2026/0232287 A1
(43) Pub. Date: Aug. 13, 2026

(54) X-RAY TUBE CALIBRATION METHOD AND DEVICE

(71) Applicant: GE Precision Healthcare LLC, Waukesha, WI (US)

(72) Inventors: Shuo Li, Beijing (CN); Yuwen Dong, Beijing (CN); Yingqing Lyu, Beijing (CN); Xueli Wang, Beijing (CN); Adam Cohen, Milwaukee, WI (US)

(21) Appl. No.: 19/152,770

(22) PCT Filed: Jan. 29, 2024
(86) PCT No.: PCT/US2024/013338

§ 371 Date: Jul. 31, 2025

(30) Foreign Application Priority Data
Jan. 31, 2023 (CN) 202310084454.0

(51) Int. Cl. A61B 6/58

(52) U.S. Cl. CPC A61B 6/582

(57) ABSTRACT

Disclosed in the present invention is an X-ray tube calibration method, including acquiring first calibration data of a replaced tube, acquiring partial calibration data of a new tube, wherein the partial calibration data is acquired by performing a scan in a particular coverage range of an X-ray beam emitted by the new tube, and calculating second complete calibration data of the new tube, wherein the calculation is performed on the basis of the first complete calibration data first calibration data and the partial calibration data. The present invention can reduce a time required for X-ray tube calibration and tube radiation life loss.

### CROSS-REFERENCE TO RELATED APPLICATIONS

This is a national stage application under 35 U.S.C. § 371 (c) of PCT Application No. PCT/US2024/013338, filed on Jan. 29, 2024, which claims priority to Chinese Application No. 202310084454.0, filed on Jan. 31, 2023, the disclosures of which are incorporated herein by reference in their entirety.

### TECHNICAL FIELD

The present invention relates to medical imaging apparatuses, and more particularly, to an X-ray tube calibration method and device for a medical imaging apparatus.

### BACKGROUND

Medical imaging apparatuses are for acquiring an anatomical structure of a scanned object, and include apparatuses for utilizing X-rays to perform medical imaging, e.g., computed tomography (CT), a digital X-ray machine, a C-arm X-ray machine, a digital subtraction angiography X-ray machine, a mammography X-ray machine, etc. For example, when a CT apparatus performs a scan, an X-ray tube emits an X-ray. The X-ray passes a test object and attenuates. A detector receives the attenuated X-ray, and converts the same into an electrical signal. After a series of processing, a computer reconstructs medical tomographic images for diagnostic reference.

After the X-ray tube of the medical imaging apparatus is replaced or after the medical imaging apparatus has been used for a period of time, the tube, especially a new X-ray tube, needs to be calibrated. X-ray tube calibration is intended to generate calibration data according to preset scan conditions, and the calibration data is for correcting an acquired medical image in a subsequent image reconstruction process, so as to improve quality of the medical image, for example, to reduce artifacts in the medical image. Currently, a commonly used X-ray tube calibration method is to perform exposure tests under a plurality of sets of preset scan conditions. In the X-ray tube calibration method, a large quantity of exposure needs to be performed, thus, a relatively long time is required to perform tests under respective scan conditions, and the large quantity of exposure would also affect the service life of the X-ray tube.

It should be noted that the above description of the background is only for the convenience of clearly and completely describing the technical solutions of the present application, and for the convenience of understanding of those skilled in the art.

### SUMMARY

In an aspect of the present invention, provided is an X-ray tube calibration method, comprising: acquiring first calibration data of a replaced tube; acquiring partial calibration data of a new tube, wherein the partial calibration data is acquired by performing a calibration scan in a particular coverage range of an X-ray beam emitted by the new tube; and calculating second complete calibration data of the new tube, wherein the calculation is performed on the basis of the first calibration data and the partial calibration data.

In another aspect of the present invention, provided is an X-ray tube calibration method, comprising: acquiring historical first calibration data of a calibrated tube; acquiring current partial calibration data of the calibrated tube, wherein the partial calibration data is acquired by performing a calibration scan in a particular coverage range of an X-ray beam emitted by the calibrated tube; and calculating second complete calibration data of the calibrated tube, wherein the calculation is performed on the basis of the first calibration data and the partial calibration data.

In another aspect of the present invention, provided is an X-ray tube calibration device, comprising: a first calibration data acquisition unit, for acquiring historical first calibration data of a replaced tube or a calibrated tube; a partial calibration data acquisition unit, for acquiring partial calibration data acquired by performing a scan in a particular coverage range of an X-ray beam emitted by a new tube or the calibrated tube; and a second complete calibration data calculation unit, for calculating second complete calibration data on the basis of the partial calibration data of the new tube and the first calibration data of the replaced tube or on the basis of the partial calibration data of the calibrated tube and the historical first calibration data thereof.

In another aspect of the present invention, provided is a computed tomography apparatus, comprising: a computed tomography scanner, comprising a tube for emitting an X-ray; and a computer, for performing any X-ray tube calibration method described above.

In another aspect of the present invention, provided is a computer-readable medium, having instructions thereon, wherein when executed by a processor, the instructions cause the processor to perform the steps of any X-ray tube calibration method described above.

These and other features and aspects of the present invention will become clearer through the detailed description with reference to the accompanying drawings below.

### BRIEF DESCRIPTION OF THE DRAWINGS

To obtain a better understanding of the present invention in detail, please refer to the embodiments for a more detailed description of the present invention as briefly summarized above. Some embodiments are illustrated in the drawings. In order to facilitate a better understanding, the same symbols have been used as much as possible in the figures to mark the same elements that are common in the various figures. It should be noted, however, that the drawings only illustrate the typical embodiments of the present invention and should therefore not be construed as limiting the scope of the present invention as the present invention may allow other equivalent embodiments. In the figures:

FIG. 1 shows a schematic system diagram of a CT apparatus according to an exemplary embodiment of the present invention.

FIG. 2 shows a system block diagram of a CT apparatus according to an exemplary embodiment of the present invention.

FIG. 3 shows a flowchart of an X-ray tube calibration method according to another exemplary embodiment of the present invention.

FIG. 4 shows a flowchart of calculating a second complete calibration parameter in an X-ray tube calibration method according to another exemplary embodiment of the present invention.

FIG. 5 shows a flowchart of an X-ray tube calibration method according to still another exemplary embodiment of the present invention.

FIG. 6 shows a flowchart of calculating a second complete calibration parameter in an X-ray tube calibration method according to still another exemplary embodiment of the present invention.

FIG. 7 shows a block diagram of an X-ray tube calibration device according to an exemplary embodiment of the present invention.

FIG. 8 shows a block diagram of a second complete calibration data calculation unit in an X-ray tube calibration device according to an exemplary embodiment of the present invention.

It can be expected that the elements in one embodiment of the present invention may be advantageously applied to the other embodiments without further elaboration.

### DETAILED DESCRIPTION

Specific implementations of the present invention will be described below. It should be noted that in the specific description of these embodiments, for the sake of brevity and conciseness, this specification may not describe all features of the actual implementations in detail. It should be understood that in the actual implementation process of any implementations, just as in the process of any engineering project or design project, a variety of specific decisions are often made to achieve specific goals of the developer and to meet system-related or business-related constraints, which may also vary from one implementation to another. Moreover, it can also be understood that although the efforts made in such development process may be complex and lengthy, for those of ordinary skill in the art related to content disclosed in the present invention, some changes in design, manufacturing, production or the like based on the technical content disclosed in the present invention are only conventional technical means. The content of the present invention should not be construed as insufficient.

Unless defined otherwise, technical terms or scientific terms used in the claims and specification should have usual meanings understood by those of ordinary skill in the technical field to which the present invention belongs. In the embodiments of the present application, the terms “first”, “second”, etc. are used to distinguish different elements, but do not represent a spatial arrangement or temporal order etc. of these elements, and these elements should not be limited by these terms. The term “and/or” includes any one of and all combinations of one or more associated listed terms. The terms “comprise”, “include”, “have”, etc. refer to the presence of described features, elements, components, or assemblies, but do not exclude the presence or addition of one or more other features, elements, components, steps, or assemblies.

In the embodiments of the present application, the singular forms “a”, “the”, etc. include plural forms, and should be broadly construed as “a type of” or “a class of” rather than limited to the meaning of “one.” Furthermore, the term “said” should be construed as including both the singular and plural forms, unless otherwise specified in the context. In addition, the term “according to” should be construed as “at least in part according to . . . ”, and the term “based on” should be construed as “at least in part based on . . . ”, unless otherwise specified in the context.

The X-ray tube calibration method described in this specification can be applied to various medical imaging apparatuses, including apparatuses that utilize X-rays to perform medical imaging, e.g., computed tomography (CT), a digital X-ray machine, a C-arm X-ray machine, a digital subtraction angiography X-ray machine, a mammography X-ray machine, a positron emission tomography-computed tomography apparatus (PET-CT), etc. The X-ray tube calibration method may be implemented by a medical imaging apparatus, or may be implemented by a separate calibration apparatus connected to a medical imaging apparatus, or may be implemented a local or remote computer that communicates with a medical imaging apparatus via the Internet. For example, the X-ray tube calibration method may be a software as a service (SaaS) application on a cloud computing platform, and a medical imaging apparatus is connected to the Internet in a wired or wireless manner. When X-ray tube calibration is implemented, a remote cloud platform computer communicates with the medical imaging apparatus, acquires imaging data of a calibration scan, then runs an X-ray tube calibration application, and returns calibration data to the medical imaging apparatus. The X-ray tube calibration apparatus described in this specification may be the medical imaging apparatus described above, and X-ray tube calibration is a function of the medical imaging apparatus. The X-ray tube calibration apparatus may also be a separate calibration apparatus, e.g., a separate local or remote computer or a handheld calibration apparatus, and the separate calibration apparatus can communicate with a medical imaging apparatus to acquire calibration scan data, and provide calibration data to the medical imaging apparatus.

The foregoing and other features of the embodiments of the present application will become apparent from the following description of this specification with reference to the accompanying drawings. In the description and the accompanying drawings, specific implementations of the present application are specifically disclosed, and part of the implementations in which the principles of the embodiments of the present application may be employed are indicated. It should be understood that the present application is not limited to the described implementations. On the contrary, the embodiments of the present application include all modifications, variations, and equivalents falling within the scope of the appended claims.

As an example, the embodiments of the present application are described below in conjunction with an X-ray computed tomography (CT) imaging apparatus. Those skilled in the art will appreciate that the embodiments of the present application can also be applied to other medical imaging apparatuses.

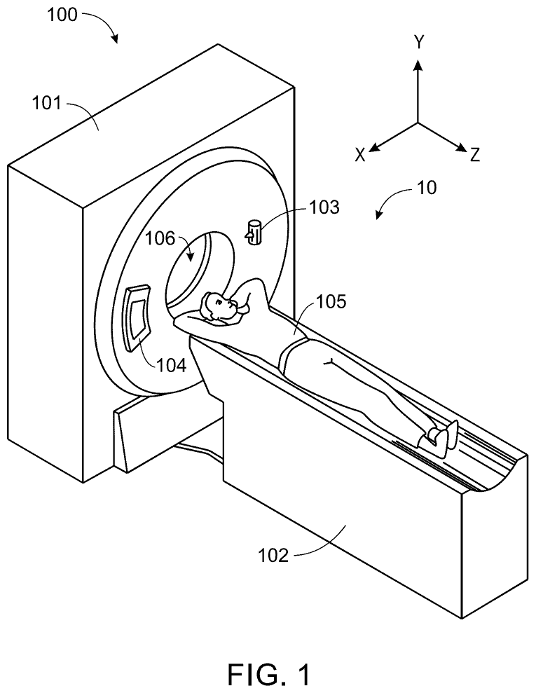

FIG. 1 is a schematic diagram of a CT imaging apparatus according to an embodiment of the present application, schematically showing the profile of a CT imaging apparatus 100. As shown in FIG. 1, the CT imaging apparatus 100 includes a scanning gantry 101 and a patient table 102. The scanning gantry 101 has an X-ray tube 103. The X-ray tube 103 projects an X-ray beam towards a detector assembly or collimator 104 on an opposite side of the scanning gantry 101. A test object 105 can lie flat on the patient table 102 and be moved into a scanning gantry opening 106 along with the patient table 102. Medical image data of the test object 105 can be acquired by means of a scan carried out by the X-ray tube 103.

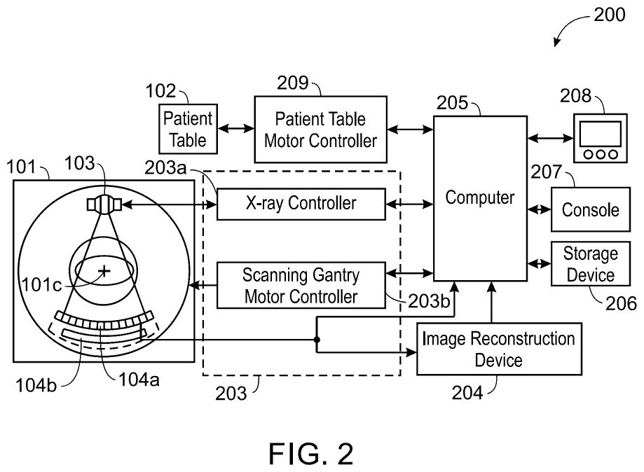

FIG. 2 is another schematic diagram of a CT imaging apparatus according to an embodiment of the present application, schematically showing a block diagram of a CT imaging apparatus 200. As shown in FIG. 2, the detector assembly 104 includes a plurality of detector units 104a and a data acquisition system (DAS) 104b. The plurality of detector units 104a sense a projected X-ray passing through the test object 105.

The DAS 104b converts, according to the sensing of the detector units 104a, collected information into projection data for subsequent processing. During the scanning for acquiring the X-ray projection data, the scanning gantry 101 and components mounted thereon rotate around a rotation center 101c.

Rotation of the scanning gantry 101 and operation of the X-ray tube 103 are controlled by a control mechanism 203 of the CT imaging apparatus 200. The control mechanism 203 includes an X-ray controller 203a that provides power and a timing signal to the X-ray tube 103 and a scanning gantry motor controller 203b that controls the rotation speed and position of the scanning gantry 101. An image reconstruction device 204 receives the projection data from the DAS 104b and performs image reconstruction. A reconstructed image is transmitted as an input to a computer 205, and the computer 205 stores the image in a mass storage device 206.

The computer 205 also receives commands and scanning parameters from an operator by means of a console 207. The console 207 has an operator interface in a certain form, such as a keyboard, a mouse, a voice activated controller, or any other suitable input device. An associated display 208 allows the operator to observe the reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 used to control the patient table 102 so as to position the test object 105 and the scanning gantry 101. In particular, the patient table 102 moves the test object 105 in whole or in part to pass through the scanning gantry opening 106 in FIG. 1.

The medical imaging apparatus is schematically illustrated above, and the X-ray tube calibration method and device according to the embodiments of the present application are described in detail below with reference to the accompanying drawings.

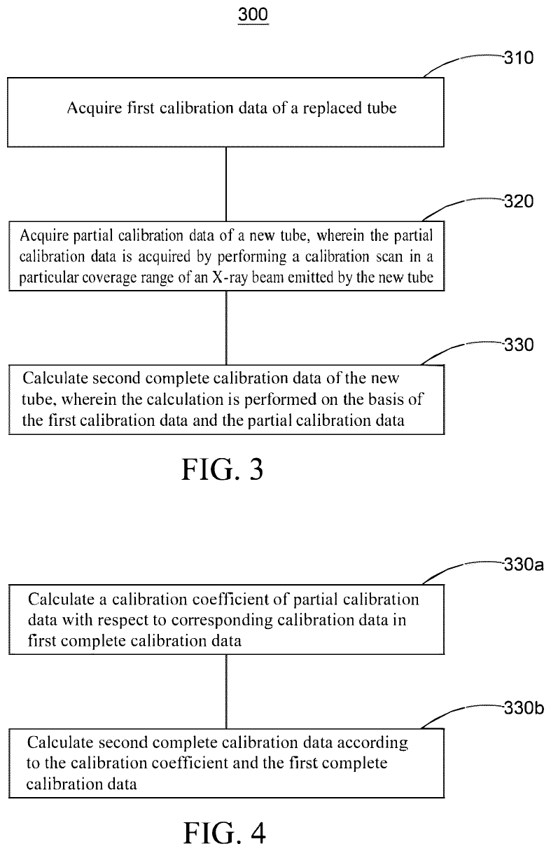

Provided in an embodiment of the present application is an X-ray tube calibration method for calibrating a replaced X-ray tube in a medical imaging apparatus after replacement. In an exemplary embodiment shown in FIG. 3, an X-ray tube calibration method 300 includes: step 310, acquiring first calibration data of a replaced tube; step 320, acquiring partial calibration data of a new tube, wherein the partial calibration data is acquired by performing a scan in a particular coverage range of an X-ray beam emitted by the new tube; and step 330, calculating second complete calibration data of the new tube, wherein the calculation is performed on the basis of the first calibration data and the partial calibration data.

It should be noted that the above description and FIG. 3 merely schematically illustrate the embodiment of the present application, but the present application is not limited thereto. For example, some other operations may also be added or some of these operations may be omitted. Those skilled in the art could make appropriate variations according to the above disclosure, rather than being limited by the disclosure of FIG. 3. It should be further noted that the “steps” in the above description and FIG. 3 represent only such different steps, but do not indicate that such steps are performed in a sequential order. For example, step 310 and step 320 may be performed concurrently. Alternatively, step 310 is performed before step 320. Alternatively, step 320 is performed before step 310.

In the above exemplary embodiment, in step 310, acquiring first calibration data of a replaced tube, wherein the first calibration data is calibration data for use of the replaced tube before the replaced tube is replaced. The calibration data includes a set of calibration parameters of the X-ray tube under all calibration scan conditions. The calibration data may be recorded in the form of a table, a vector, or other suitable data forms. As an example, the calibration scan conditions include factors of four aspects, i.e., a tube voltage, a collimator aperture width, the size of a ray shape filter, and the size of a tube focal spot. The tube voltage is a high voltage applied to the X-ray tube. When a calibration scan is performed, the tube voltages may include 70 kVp, 80 kVp, 100 kVp, 120 kVp, and 140 kVp. A collimator is located at an outlet of the X-ray of the X-ray tube and before a scanned object, and is for adjusting the size of a fan-beam or cone-beam X-ray emitted by the X-ray tube. The collimator aperture width is the size of an opening of an X-ray shielding gate of the collimator in a direction (the Z direction shown in FIG. 1) in which a patient enters or exits a CT imaging apparatus, i.e., the size in a widthwise direction of an X-ray beam. The CT imaging apparatus adjusts the coverage range of the X-ray tube by adjusting the value of the collimator aperture width. That is, the collimator aperture width represents the coverage range of the X-ray beam emitted by the tube. When a calibration scan is performed, the collimator aperture width may be a plurality of discrete values between 5 mm and 160 mm. The ray shape filter is for manipulating and further changing spectral or spatial intensity distribution of X-ray radiation. For example, a bowtie filter additionally causes X-ray radiation to be focused or expanded by means of a protruding or recessed face. Typically, ray shape filters are divided into three types, i.e., large, medium, and small ray shape filters. The sizes of focal spots may be divided into three types, i.e., large, medium, and small sizes. The calibration scan conditions are combinations of such four aspects of factors. For example, an X-ray tube calibration parameter is acquired after scans are performed by the CT imaging apparatus under the calibration scan conditions: the tube voltage being 70 kVp, the collimator aperture width being 5 mm, the before-patient ray filter being large, and the focal spot being small. As an example, the first calibration data includes complete calibration data acquired by performing scans under all calibration scan conditions of the replaced tube. That is, the first calibration data is acquired by performing scans under 405 calibration scan conditions being combinations of the tube voltage, the collimator aperture width, the size of the ray shape filter, and the size of the focal spot. In some examples, the first calibration data may be acquired by performing scans under some of the calibration scan conditions, and calibration data under the calibration scan conditions other than the foregoing some of the calibration scan conditions is acquired by means of mathematical calculation. Then, the two parts of data are combined into complete calibration data. In some other examples, the first calibration data may include only calibration data acquired by performing scans under some of the calibration scan conditions.

In the above exemplary embodiment, in step 320, acquiring partial calibration data of a new tube, wherein the partial calibration data is acquired by performing a scan in a particular coverage range of the new tube. As an example, the particular coverage range of the new tube is a maximum coverage range of the tube, that is, a maximum value of the collimator aperture width. As an example, in a CT imaging apparatus having 256 rows of detectors, the detector width is 160 mm. Correspondingly, the new tube is configured to have the coverage range of 160 mm. That is, the calibration scan is performed by increasing the collimator aperture width to the maximum value, i.e., 160 mm. When a calibration scan is performed in this case, the new tube emits the X-ray under the scan condition of each tube voltage, size of the focal spot, and filter type. After the detector receives the X-ray, a set of scan data is formed, so that the partial calibration data of the new tube is acquired. As an example, a set of scans including totally 45 scans are respectively performed under conditions: the coverage range of the new tube being 160 mm, the tube voltages being 70 kVp, 80 kVp, 100 kVp, 120 kVp, and 140 kVp, the sizes of the focal spot being large, medium, and small, and the ray shape filters being large, medium, and small. In the foregoing embodiment, the calibration scan is performed in the maximum coverage range of the new tube. The X-ray emitted by the new tube can reach all detection units of the detector, and calibration data of all of the detection units of the detector can be acquired by means of a set of calibration scans. In other examples, the particular coverage range of the new tube may be smaller than the foregoing maximum coverage range, and may be, e.g., 100 mm and 120 mm. In the foregoing example, the particular coverage range of the new tube is a single range. In other examples, the particular coverage range of the new tube may include a plurality of ranges, e.g., 40 mm and 160 mm. When tube calibration is performed, two sets of calibration scans are respectively performed in the two coverage ranges, thereby acquiring the partial calibration data.

In the above exemplary embodiment, in step 330, calculating second complete calibration data of the new tube, wherein the calculation is performed on the basis of the first calibration data and the partial calibration data. As shown in FIG. 4, the calculating second complete calibration data of the new tube includes: step 330a, calculating a calibration coefficient of the partial calibration data with respect to corresponding calibration data in the first calibration data; and step 330b, calculating the second complete calibration data according to the calibration coefficient and the first calibration data.

As an example, in step 330a, the calibration coefficient is the quotient of the partial calibration data and the corresponding calibration data in the first calibration data. For the calculation of the calibration coefficient, reference can be made to Equation (1-1):

$$( Cal ) coefficient = \frac{{( Cal )}_{{aperature}_{new}}}{{( Cal )}_{{aperature}_{old}}} \qquad Equation ( 1 - 1 )$$

where (Cal)coefficient represents the calibration coefficient, and (Cal)ₐₚₑᵣₜᵤᵣₑ_{_{new}} represents the partial calibration data acquired by performing the calibration scan in the particular coverage range of the new tube, wherein aperture represents the coverage range of the tube, i.e., the collimator aperture width of the tube, and (Cal)ₐₚₑᵣₜᵤᵣₑ_{_{old}} represents the calibration data of the replaced tube in the foregoing particular coverage range, which corresponds to the partial calibration data of the new tube.

As an example, for the tube with a maximum radiation range of 160 mm, the calibration coefficient can be acquired by dividing the partial calibration data acquired by performing the calibration scan in the radiation range of 160 mm of the new tube by the calibration data of the replaced tube in the radiation range of 160 mm thereof. Refer to the following Equation (1-2) for the calculation of the calibration coefficient:

$$( Cal ) coefficient = \frac{{( Cal )}_{{160}_{new}}}{{( Cal )}_{{160}_{old}}} \qquad Equation ( 1 - 2 )$$

where (Cal)coefficient represents the calibration coefficient, and Cal₁₆₀_{_{new}} represents the partial calibration data acquired by performing the calibration scan in the radiation range of 160 mm of the new tube. Cal₁₆₀_{_{old}} represents the calibration data of the replaced tube in the radiation range of 160 mm thereof, which corresponds to the partial calibration data of the new tube. In this example, the particular coverage range is the maximum coverage range of the new tube. In other examples, the particular coverage range may be smaller than the maximum coverage range, e.g., a coverage range of 140 mm. In other examples, a small number of particular coverage ranges, e.g., two particular coverage ranges, may be selected. The partial calibration data includes first partial calibration data and second calibration data acquired by respectively performing scans in different particular coverage ranges of the new tube. The calculating second complete calibration data of the new tube includes: respectively calculating a first calibration coefficient and a second calibration coefficient of the first partial calibration data and the second calibration data with respect to the corresponding calibration data in the first calibration data. The first calibration coefficient and the second calibration coefficient are combined into the calibration coefficient. For example, an average value, or a root mean square, or another suitable calculation result of the first calibration coefficient and the second calibration coefficient is calculated as the calibration coefficient.

As an example, in step 330b, the second complete calibration data of the new tube is the product of the calibration coefficient and the first calibration data. Refer to the following Equation (1-3) for the calculation of the second complete calibration data:

$${( Cal )}_{{aperature}_{new}} = ( Cal ) coefficient \times {( Cal )}_{{aperature}_{old}} \qquad Equation ( 1 - 3 )$$

where (Cal) coefficient represents the calibration coefficient calculated according to formula (1-1), and (Cal)ₐₚₑᵣₜᵤᵣₑ_{_{new}} represents the calibration data of the new tube in the particular radiation range, wherein aperture represents the coverage range of the tube, i.e., the collimator aperture width of the tube. (Cal)ₐₚₑᵣₜᵤᵣₑ_{_{old}} represents the calibration data of the replaced tube in the foregoing particular coverage range.

When the second complete calibration data is calculated, the calibration data of the first calibration data in each particular coverage range of the tube can be multiplied by the calibration coefficient, so as to acquire the calibration data of the new tube in each particular coverage range of the tube. As an example, calibration data of the new tube in the radiation range of 40 mm thereof can be the calibration coefficient multiplied by calibration data of the replaced tube in the radiation range of 40 mm thereof. For the calculation, reference can be made to the following Equation (1-4):

$${( C a l )}_{4 {0}_{n e w}} = Cal coefficient \times {( C a l )}_{4 {0}_{o l d}} \qquad Equation ( 1 - 4 )$$

where Cal coefficient represents the calibration coefficient. (Cal)₄₀_{_{new}} represents the calibration data of the replaced tube in the radiation range of 40 mm thereof, and (Cal)₄₀_{_{old}} represents the calibration data of the replaced tube in the radiation range of 40 mm thereof.

Calibration data of the new tube in other particular radiation ranges, such as 80 mm, 120 mm, and 140 mm, can also be the calibration coefficient multiplied by corresponding calibration data of the replaced tube in such particular radiation ranges, so as to acquire the second complete calibration data of the new tube.

In the foregoing embodiment, the calibration coefficient is the quotient of the partial calibration data and the corresponding calibration data in the first calibration data, and correspondingly, the second complete calibration data of the new tube is the product of the calibration coefficient and the first calibration data.

As an example, the X-ray tube calibration method is an air calibration method. No scanned object is placed in a scan space of the CT imaging apparatus when a calibration scan is performed. That is, an X-ray passes through air, and reaches the detector. The X-ray tube calibration method can also be applied to other suitable X-ray tube calibration.

In the foregoing example, the partial calibration data of the new tube is acquired, and the partial calibration data is acquired by performing the scan in the particular coverage range of the new tube. The second complete calibration data of the new tube is calculated, and the calculation is performed on the basis of the first calibration data and the partial calibration data. One set of or a small number of sets of calibration scans are performed for the new tube, and calibration scans do not need to be performed for all particular ranges, thereby conserving a calibration time and reducing tube radiation life loss.

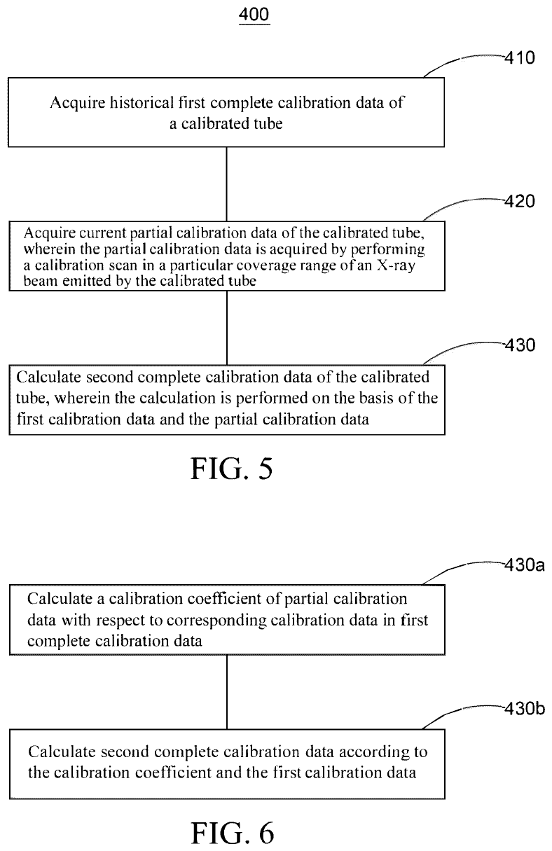

Provided in an embodiment of the present application is an X-ray tube calibration method for calibrating an X-ray tube regularly after a medical imaging apparatus has been used for a period of time. In an exemplary embodiment shown in FIG. 5, an X-ray tube calibration method 400 includes: step 410, acquiring historical first calibration data of a calibrated tube; step 420, acquiring current partial calibration data of the calibrated tube, wherein the partial calibration data is acquired by performing a calibration scan in a particular coverage range of an X-ray beam emitted by the calibrated tube; and step 430, calculating second complete calibration data of the calibrated tube, wherein the calculation is performed on the basis of the first calibration data and the partial calibration data.

It should be noted that the above description and FIG. 5 merely schematically illustrate the embodiment of the present application, but the present application is not limited thereto. For example, some other operations may also be added or some of these operations may be omitted. Those skilled in the art could make appropriate variations according to the above disclosure, rather than being limited by the disclosure of FIG. 5. It should be further noted that the “steps” in the above description and FIG. 5 represent only such different steps, but do not indicate that such steps are performed in a sequential order. For example, step 410 and step 420 may be performed concurrently. Alternatively, step 410 is performed before step 420. Alternatively, step 420 is performed before step 410.

In the above exemplary embodiment, in step 410, acquiring historical first calibration data of a calibrated tube, wherein the first calibration data is calibration data for use of the calibrated tube before current calibration. The calibration data includes a set of calibration parameters of the X-ray tube under all calibration scan conditions. The calibration data may be recorded in the form of a table, a vector, or other suitable data forms. As an example, similar to the first calibration data in the embodiments of the first aspect. The first calibration data includes complete calibration data acquired by performing scans under all calibration scan conditions of the calibrated tube. That is, the first calibration data is acquired by performing scans under 405 calibration scan conditions being combinations of the tube voltage, the collimator aperture width, the size of the ray shape filter, and the size of the focal spot. In some examples, the first calibration data may be acquired by performing scans under some of the calibration scan conditions, and calibration data under the calibration scan conditions other than the foregoing some of the calibration scan conditions is acquired by means of mathematical calculation. Then, the two parts of data are combined into complete calibration data. In some other examples, the first calibration data may include only calibration data acquired by performing scans under some of the calibration scan conditions.

In the above exemplary embodiment, in step 420, acquiring current partial calibration data of the calibrated tube, wherein the partial calibration data is acquired by performing a calibration scan in a particular coverage range of an X-ray beam emitted by the calibrated tube. As an example, the particular coverage range of the calibrated tube is a maximum coverage range of the tube, that is, a maximum value of the collimator aperture width. As an example, in a CT imaging apparatus having 256 rows of detectors, the detector width is 160 mm. Correspondingly, the calibrated tube is configured to have the coverage range of 106 mm. That is, the calibration scan is performed by increasing the collimator aperture width to the maximum value, i.e., 160 mm.

When a calibration scan is performed in this case, the calibrated tube emits the X-ray under the scan condition of each tube voltage, size of the focal spot, and filter type. After the detector receives the X-ray, a set of scan data is formed, so that the partial calibration data of the new tube is acquired. As an example, a set of scans including totally 45 scans are respectively performed under conditions: the coverage range of the calibrated tube being 160 mm, the tube voltages being 70 kVp, 80 kVp, 100 kVp, 120 kVp, and 140 kVp, the sizes of the focal spot being large, medium, and small, and the ray shape filters being large, medium, and small. In the foregoing embodiment, the calibration scan is performed in the maximum coverage range of the calibrated tube. The X-ray emitted by the calibrated tube can reach all detection units of the detector, and calibration data of all of the detection units of the detector can be acquired by means of a set of calibration scans. In other examples, the particular coverage range of the calibrated tube may be smaller than the foregoing maximum coverage range, and may be, e.g., 100 mm and 120 mm. In the foregoing example, the particular coverage range of the new tube is a single range. In other examples, the particular coverage range of the new tube may include a plurality of ranges, e.g., 40 mm and 160 mm. When tube calibration is performed, two sets of calibration scans are respectively performed in the two coverage ranges, thereby acquiring the partial calibration data.

In the above exemplary embodiment, in step 430, calculating second complete calibration data of the calibrated tube, wherein the calculation is performed on the basis of the foregoing first calibration data and the foregoing partial calibration data. As shown in FIG. 6, the calculating second complete calibration data of the calibrated tube includes: step 430a, calculating a calibration coefficient of the foregoing partial calibration data with respect to corresponding calibration data in the foregoing first calibration data; and step 430b, calculating the second complete calibration data according to the calibration coefficient and the first calibration data. As an example, the calibration coefficient is the quotient of the foregoing partial calibration data and the corresponding calibration data in the foregoing first calibration data. For the calculation of the calibration coefficient, reference can be made to Equation (2-1)

$$( Cal ) coefficient = \frac{{( Cal )}_{{aperature}_{current}}}{{( Cal )}_{{aperature}_{historical}}} \qquad Equation ( 2 - 1 )$$

where (Cal)coefficient represents the calibration coefficient, and (Cal)ₐₚₑᵣₜᵤᵣₑ_{_{current}} represents the current partial calibration data acquired by performing the calibration scan in the particular coverage range of the calibrated tube, wherein aperture represents the coverage range of the tube, i.e., the collimator aperture width of the tube, and (Cal)ₐₚₑᵣₜᵤᵣₑ_{_{historical}} represents the historical calibration data of the calibrated tube in the foregoing particular coverage range, which corresponds to the current partial calibration data of the calibrated tube.

As an example, for the tube with a maximum radiation range of 160 mm, the calibration coefficient can be acquired by dividing the current partial calibration data acquired by performing the calibration scan in the radiation range of 160 mm of the calibrated tube by the historical calibration data of the calibrated tube in the radiation range of 160 mm thereof. Refer to the following Equation (2-2) for the calculation of the calibration coefficient:

$$( Cal ) coefficient = \frac{{( Cal )}_{{160}_{current}}}{{( Cal )}_{{160}_{historical}}} \qquad Equation ( 2 - 2 )$$

where (Cal)coefficient represents the calibration coefficient, and (Cal)₁₆₀_{_{current}} represents the current partial calibration data acquired by performing the calibration scan in the radiation range of 160 mm of the calibrated tube. (Cal)₁₆₀_{_{historical}} represents the historical calibration data of the calibrated tube in the radiation range of 160 mm thereof, which corresponds to the current partial calibration data of the calibrated tube. In this example, the particular coverage range is the maximum coverage range of the calibrated tube. In other examples, the particular coverage range may be a coverage range smaller than the maximum coverage range, e.g., a coverage range of 140 mm. In other examples, a small number of particular coverage ranges, e.g., two particular coverage ranges, may be selected. The partial calibration data includes first partial calibration data and second partial calibration data acquired by respectively performing calibration scans in different particular coverage ranges of the calibrated tube. The calculating second complete calibration data of the new tube includes: respectively calculating a first calibration coefficient and a second calibration coefficient of the first partial calibration data and the second partial calibration data with respect to the corresponding calibration data in the first calibration data. The first calibration coefficient and the second calibration coefficient are combined into the calibration coefficient. For example, an average value, or a root mean square, or another suitable calculation result of the first calibration coefficient and the second calibration coefficient is calculated as the calibration coefficient.

As an example, the second complete calibration data of the calibrated tube is the product of the calibration coefficient and the first calibration data. Refer to the following Equation (2-3) for the calculation of the second complete calibration data:

$${( Cal )}_{{aperature}_{current}} = ( Cal ) coefficient \times {( Cal )}_{{aperature}_{historical}} \qquad Equation ( 2 - 3 )$$

where (Cal)coefficient represents the calibration coefficient calculated according to formula (2-1), and (Cal)ₐₚₑᵣₜᵤᵣₑ_{_{current}} represents the current calibration data of the calibrated tube in the particular radiation range, wherein aperture represents the coverage range of the tube, i.e., the collimator aperture of the tube. (Cal)ₐₚₑᵣₜᵤᵣₑ_{_{historical}} represents the historical calibration data of the calibrated tube in the foregoing particular coverage range.

When the second complete calibration data is calculated, the calibration data of the first calibration data in each particular coverage range of the tube can be multiplied by the calibration coefficient, so as to acquire the calibration data of the new tube in each coverage range of the tube. As an example, current calibration data of the calibrated tube in the radiation range of 40 mm can be the calibration coefficient multiplied by historical calibration data of the calibrated tube in the radiation range of 40 mm. For the calculation, reference can be made to the following Equation (2-4):

$${( C a l )}_{4 {0}_{current}} = ( Cal ) coefficient \times {( C a l )}_{4 {0}_{historical}} \qquad Equation ( 2 - 4 )$$

where (Cal)coefficient represents the calibration coefficient. (Cal)₄₀_{_{current}} represents the current calibration data of the calibrated tube in the radiation range of 40 mm, and (Cal)₄₀_{_{historical}} represents the historical calibration data of the calibrated tube in the radiation range of 40 mm.

Current calibration data of the calibrated tube in other particular radiation ranges, such as 80 mm, 120 mm, and 140 mm, can also be the calibration coefficient multiplied by corresponding historical calibration data of the calibrated tube in such particular radiation ranges, so as to acquire the second complete calibration data of the calibrated tube.

In the foregoing embodiment, the calibration coefficient is the quotient of the current partial calibration data of the calibrated tube and the corresponding calibration data in the historical first calibration data thereof, and correspondingly, the current second complete calibration data of the calibrated tube is the product of the calibration coefficient and the historical first calibration data thereof.

As an example, the X-ray tube calibration method is an air calibration method. No scanned object is placed in a scan space of the CT imaging apparatus when a calibration scan is performed. That is, an X-ray passes through air, and reaches the detector. The X-ray tube calibration method can also be applied to other suitable X-ray tube calibration.

In the foregoing example, the current partial calibration data of the calibrated tube is acquired, and the partial calibration data is acquired by performing the calibration scan in the particular coverage range of the calibrated tube. The current second complete calibration data of the calibrated tube is calculated, and the calculation is performed on the basis of the foregoing historical first calibration data and the current partial calibration data. One set of or a small number of sets of calibration scans are performed for the calibrated tube, and calibration scans do not need to be performed for all particular ranges, thereby conserving a calibration time and conserving the tube radiation life.

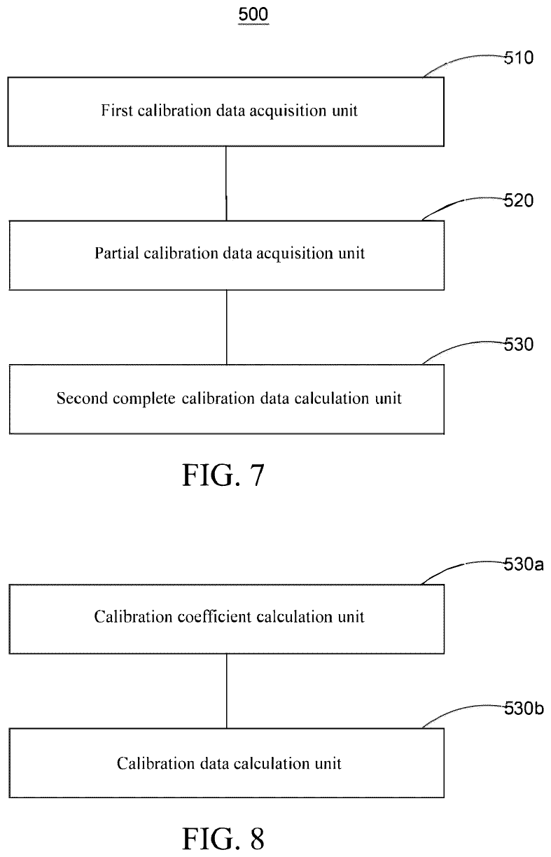

Further provided in an embodiment of the present application is an X-ray tube calibration device for regularly calibrating a new X-ray tube after tube replacement or an X-ray tube having been used for a period of time. In an exemplary embodiment shown in FIG. 7, the X-ray tube calibration device includes: a first calibration data acquisition unit 510, for acquiring historical first calibration data of a replaced tube or a calibrated tube; a partial calibration data acquisition unit 520, for acquiring partial calibration data acquired by performing a calibration scan in a particular coverage range of a new tube or the calibrated tube; and a second complete calibration data calculation unit 530, for calculating second complete calibration data on the basis of the partial calibration data of the new tube and the first calibration data of the replaced tube or on the basis of the partial calibration data of the calibrated tube and the historical first calibration data thereof.

In the above exemplary embodiment, the first calibration data acquisition unit 510 acquires historical first calibration data of a replaced tube or a calibrated tube. As an example, in a scenario in which calibration is performed after an X-ray tube of a medical imaging apparatus is replaced, the first calibration data acquisition unit 510 acquires complete calibration data of the replaced X-ray tube, i.e., complete calibration data acquired by performing scans under all calibration scan conditions of the replaced tube. In a scenario in which an X-ray tube is calibrated regularly after a medical imaging apparatus has been used for a period of time, the first calibration data acquisition unit 510 acquires historical first calibration data of the calibrated X-ray tube, i.e., calibration data of the calibrated X-ray tube used in scans before current calibration. As an example, the first calibration data is historical complete calibration data of the calibrated tube, i.e., historical complete calibration data acquired by performing calibration scans under all calibration scan conditions of the calibrated tube before the current calibration.

In the above exemplary embodiment, the partial calibration data acquisition unit 520 acquires partial calibration data acquired by performing a scan in a particular coverage range of a new tube or the calibrated tube. In the scenario in which calibration is performed after an X-ray tube of a medical imaging apparatus is replaced, the partial calibration data acquisition unit 520 performs step 320 according to the foregoing embodiment of the first aspect. In the scenario in which an X-ray tube is calibrated regularly after a medical imaging apparatus has been used for a period of time, the partial calibration data acquisition unit 520 performs step 420 according to the foregoing embodiment of the second aspect.

In the above exemplary embodiment, the second complete calibration data calculation unit 530 calculates second complete calibration data on the basis of the partial calibration data of the new tube and the first calibration data of the replaced tube or on the basis of the partial calibration data of the calibrated tube and the historical first calibration data thereof. As an example, the second complete calibration data calculation unit 530 includes a calibration coefficient calculation unit 530a and a calibration data calculation unit 530b as shown in FIG. 8.

In the scenario in which calibration is performed after an X-ray tube of a medical imaging apparatus is replaced, the calibration coefficient calculation unit 530a calculates the calibration coefficient according to step 330a of the foregoing embodiment of the first aspect on the basis of the partial calibration data acquired by the partial calibration data acquisition unit 520 and the first calibration data acquired by the first calibration data acquisition unit 510. As an example, the calibration coefficient is the quotient of the partial calibration data and the corresponding calibration data in the first calibration data. The calibration data calculation unit 530b calculates the second complete calibration data according to step 330b of the foregoing embodiment of the first aspect. As an example, the calibration data calculation unit 530b calculates the product of the calibration coefficient and the calibration data of the replaced tube in each coverage range, so as to acquire the calibration data of the new tube in each particular coverage range thereof, thereby forming the second complete calibration data.

In the scenario in which an X-ray tube is calibrated regularly after a medical imaging apparatus has been used for a period of time, the calibration coefficient calculation unit 530a calculates the calibration coefficient according to step 430a of the foregoing embodiment of the second aspect on the basis of the partial calibration data acquired by the partial calibration data acquisition unit 520 and the historical first calibration data acquired by the first calibration data acquisition unit 510. As an example, the calibration coefficient is the quotient of the partial calibration data and the corresponding calibration data in the first calibration data. The calibration data calculation unit 530b calculates the second complete calibration data according to step 430b of the foregoing embodiment of the second aspect. As an example, the calibration data calculation unit 530b calculates the product of the calibration coefficient and the historical calibration data of the calibrated tube in each particular coverage range, so as to acquire the calibration data of the calibrated tube in each particular coverage range thereof, thereby forming the second complete calibration data.

As an example, the X-ray tube calibration apparatus described in this specification may be the medical imaging apparatus described above, and X-ray tube calibration is a function of the medical imaging apparatus. The X-ray tube calibration apparatus may also be a separate calibration apparatus, e.g., a separate local or remote computer or a handheld calibration apparatus, and the separate calibration apparatus can communicate with a medical imaging apparatus to acquire calibration scan data, and provide calibration data to the medical imaging apparatus.

Further provided in an embodiment of the present application is a computer-readable program, wherein when the program is executed, the program causes a computer to perform, in the device or system or computer apparatus, the X-ray tube calibration method described in the foregoing embodiment.

Further provided in an embodiment of the present application is a storage medium storing a computer-readable program, wherein the computer-readable program causes a computer to perform, in a device or system or computer apparatus, the X-ray tube calibration method described in the foregoing embodiment.

The above embodiments merely provide illustrative description of the embodiments of the present application. However, the present application is not limited thereto, and appropriate variations may be made on the basis of the above embodiments. For example, each of the above embodiments may be used independently, or one or more of the above embodiments may be combined.

Some exemplary embodiments of the present invention have been described above. However, it should be understood that various modifications can be made to the exemplary embodiments described above without departing from the spirit and scope of the present invention. For example, an appropriate result can be achieved if the described techniques are performed in a different order and/or if the components of the described system, architecture, apparatus, or circuit are combined in other manners and/or replaced or supplemented with additional components or equivalents thereof; accordingly, the modified other embodiments also fall within the protection scope of the claims.

1. An X-ray tube calibration method, comprising:
acquiring first calibration data of a replaced tube;
acquiring partial calibration data of a new tube, wherein the partial calibration data is acquired by performing a calibration scan in a particular coverage range of an X-ray beam emitted by the new tube; and
calculating second complete calibration data of the new tube, wherein calculating second complete calibration data is performed based on the first calibration data and the partial calibration data.

2. The method according to claim 1, wherein the first calibration data comprises complete calibration data acquired by performing scans under all calibration scan conditions of the replaced tube.

3. The method according to claim 1, wherein the partial calibration data is acquired by performing a scan in a maximum coverage range in a widthwise direction of the X-ray beam of the new tube.

4. The method according to claim 3, wherein the calculating second complete calibration data of the new tube comprises calculating a calibration coefficient of the partial calibration data with respect to corresponding calibration data in the first calibration data.

5. The method according to claim 4, wherein the calibration coefficient is a quotient of the partial calibration data and the corresponding calibration data in the first calibration data.

6. The method according to claim 5, wherein the calculating second complete calibration data of the new tube comprises calculating a product of the first calibration data and the calibration coefficient.

7. The method according to claim 1, wherein the calibration method comprises air calibration of an X-ray tube.

8. An X-ray tube calibration method, comprising:
acquiring historical first calibration data of a calibrated tube;
acquiring current partial calibration data of the calibrated tube, wherein the partial calibration data is acquired by performing a scan in a particular coverage range of an X-ray beam emitted by the calibrated tube; and
calculating second complete calibration data of the calibrated tube, wherein calculating second complete calibration data is performed based on of the first calibration data and the partial calibration data.

9. The method according to claim 8, wherein the first calibration data comprises complete calibration data acquired by performing scans under all calibration scan conditions of the calibrated tube.

10. The method according to claim 8, wherein the partial calibration data is acquired by performing a scan in a maximum coverage range in a widthwise direction of the X-ray beam of the calibrated tube.

11. The method according to claim 10, wherein the calculating second complete calibration data of the calibrated tube comprises calculating a calibration coefficient of the partial calibration data with respect to corresponding calibration data in the first calibration data.

12. The method according to claim 11, wherein the calibration coefficient is a quotient of the partial calibration data and the corresponding calibration data in the first calibration data.

13. The method according to claim 12, wherein the calculating second complete calibration data of the calibrated tube comprises calculating a product of the first calibration data and the calibration coefficient.

14. An X-ray tube calibration device, comprising:
a first calibration data acquisition unit, for acquiring historical first calibration data of a replaced tube or a calibrated tube;
a partial calibration data acquisition unit, for acquiring partial calibration data acquired by performing a scan in a particular coverage range of an X-ray beam emitted by a new tube or the calibrated tube; and
a second complete calibration data calculation unit, for calculating second complete calibration data based on the partial calibration data of the new tube and the first calibration data of the replaced tube or based on the partial calibration data of the calibrated tube and the historical first calibration data thereof.

15. The device according to claim 14, wherein the first calibration data comprises complete calibration data acquired by performing scans under all calibration scan conditions of the replaced tube or the calibrated tube.

16. The device according to claim 14, wherein the partial calibration data acquisition unit acquires partial calibration data acquired by performing a scan in a maximum coverage range in a widthwise direction of the X-ray beam of the new tube or the calibrated tube.

17. The device according to claim 14, wherein the second complete calibration data calculation unit comprises a calibration coefficient calculation unit for calculating a calibration coefficient of the partial calibration data with respect to corresponding calibration data in the first calibration data.

18. The device according to claim 17, wherein the calibration coefficient is a quotient of the partial calibration data and the corresponding calibration data in the first calibration data, and the second complete calibration data calculation unit further comprises a calibration coefficient calculation unit for calculating a product of the first calibration data and the calibration coefficient.
